(19) United States
(12) Patent Application Publication
(10) Pub. No.: US 2024/0141280 A1
(43) Pub. Date: May 2, 2024

(54) BIOLOGICAL SPECIMEN IMAGING UNIT FOR USE IN INCUBATOR

(71) Applicant: Analog Devices, Inc., Wilmington, MA (US)

(72) Inventors: Max Marcel Shulaker, Cambridge, MA (US); Samuel H. Fuller, Wellesley, MA (US)

(21) Appl. No.: 18/391,378

(22) Filed: Dec. 20, 2023

Related U.S. Application Data
(60) Provisional application No. 63/476,323, filed on Dec. 20, 2022.

(51) Int. Cl. C12M 1/34; C12M 1/00; C12M 1/22; C12M 1/36; C12M 3/00; G06V 20/69; H04N 7/18; H04N 23/90

(52) U.S. Cl. CPC C12M 41/36; C12M 23/10; C12M 23/48; C12M 41/14; C12M 41/34; C12M 41/48; G06V 20/693; H04N 7/183; H04N 23/90

(57) ABSTRACT

An imaging unit can be configured for biological specimen sensing inside an incubator. Such an imaging unit can include an imager in communication with one or more biological specimen vessels for generating a corresponding specimen image of a specimen. Processing circuitry can control the imager to generate the specimen image. This can include controlling at least one of the imager and the individual vessel to locate the vessel in a field of view of the imager for obtaining the specimen image. The specimen image can be image-processed such as to determine an indication of a biological characteristic associated with the specimen.

### CLAIM OF PRIORITY

This application claims priority to U.S. Provisional Application Ser. No. 63/476,323, filed on Dec. 20, 2022, which is incorporated by reference herein in its entirety, and the benefit of priority of which is claimed herein.

### BACKGROUND

Incubation of biological samples (such as sputum, urine, stool, or blood) can be used to help detect foreign bodies, such as bacteria, within a human patient. For example, a blood sample can be taken from the patient and incubated to determine whether an infection exists. During an incubation period, certain microorganism cultures can multiply within the blood. Incubation can help enable several techniques to detect infections. For example, an observable change in the sample's properties can develop throughout incubation. The sample can be analyzed to help form a medical diagnosis of the patient.

Incubation periods of biological samples can range between about four hours to >24 hours, depending upon the type of microorganism and the ambient temperature. The sample can be maintained at approximately body temperature (about 37° C.), or the temperature of the sample can be established such as to enhance growth of a certain target foreign body. Such incubation processes can involve combining the sample with a fluid, such as an aqueous buffered salt solution (BSS) with added nutrients, or on a solid media such as a petri dish or slant with nutrient agar. A blood sample can be separated into several components (e.g., plasma, red blood cells, white blood cells, platelets, etc.) after collection. Such separated components can be similarly incubated, observed, and analyzed. Also, other body fluids can be used for similar incubation and analysis, such as spinal fluid, synovial fluid, cerebrospinal fluid, sweat, urine, and saliva.

### SUMMARY

Certain biological specimen incubation techniques can involve one or more manual operations during or between incubation sessions. Intervening manually in the incubating of a specimen can present challenges, such as difficulty in obtaining repeatable results and possible operator-introduced errors. Further, an incubation technique can involve visual inspection of biological specimens, such as for signs of an infection. This can include observing for one or more signs of a change in color, clarity, size, or other characteristics of the specimen. Such visual inspection may introduce contamination or may lead to operator-introduced errors in determining whether a target foreign body is present within the biological specimen. The manual checking of petri dishes also limits the frequency at which incubation status can be checked, delaying the diagnosis. Moreover, removing samples from incubation can disturb the growth process, delaying the growth of the microorganisms. The present inventors have recognized a need for a more user-friendly, more reproducible, less operator-dependent, more sterile, and more cost-efficient technique for biological specimen incubation and analysis.

This document describes a specimen imaging unit that can be configured for biological specimen sensing inside an incubator. Such a specimen imaging unit can be inserted or retrofitted into an incubator. The specimen imaging unit can include an imager. The imager can be arranged to be placed inside the incubator in communication with one or more biological specimen vessels. The imager can be configured for generating a corresponding specimen image of a corresponding biological specimen. The specimen imaging unit can include or use a plurality of receptacles. An individual receptacle can be sized and shaped for receiving an individual one of the biological specimen vessels. The specimen imaging unit can also include an illuminator to illuminate the individual biological specimen vessel with electromagnetic energy. For example, the illuminator can include a broadband or tunable wavelength electromagnetic energy source.

The specimen imaging unit can include or can be communicatively coupled to processing circuitry. The processing circuitry can be configured to control the imager for generating the specimen image. For example, the processing circuitry can control at least one of the imager or the individual vessel such as to locate the vessel in a field of view of the imager for obtaining the specimen image. Once obtained, the specimen image can be processed. The image-processing can be performed to help determine an indication of a biological characteristic associated with the biological specimen. Once determined, the indication of the biological characteristic can be transmitted, such as via transceiver circuitry, to an interface device located outside the incubator. The specimen imaging unit can include a transporter communicatively coupled with the processing circuitry to move at least one of the imager or an individual biological specimen vessel with respect to the other. For example, the transporter can include a robotic manipulator for retrieving the individual vessel and placing the vessel in a field of view of the imager. Alternatively or additionally, the transporter can move the imager toward an individual receptacle of the plurality of receptacles. In an example, the specimen imaging unit can include a plurality of imaging subunits arranged within a sealed chamber. The plurality of imaging subunits can be configured for concurrently imaging respective vessels therein. For example, each imaging subunit can include a respective transporter. Alternatively or additionally, the transporter can service multiple imaging subunits each containing different respective pluralities of specimen vessels.

In an example, an individual vessel can include or be communicatively coupleable to an electrochemical transducer for transducing an electrical property, indicative of a target gas composition corresponding with the particular biological specimen, into an electrical response signal. Here, a presence of a particular biological specimen can be determined by analyzing both the specimen image and the electrical property, such as concurrently or otherwise. For example, the individual vessel can include a port such as can be communicatively coupled with a receptacle of the electrochemical transducer.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The Detailed Description is included to provide further information.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an example of a biological specimen incubator.

FIG. 1B depicts a biological specimen imaging unit for placement into a sealed region of an incubator.

FIG. 2 depicts an example of a modular biological specimen imaging unit for placement within a biological specimen incubator.

FIG. 3 is a block diagram that describes an example of a biological specimen imaging system.

FIG. 4A depicts an example of a biological specimen imaging system.

FIG. 4B depicts an example of a biological specimen imaging system.

FIG. 4C depicts an example of a biological specimen imaging system.

FIG. 5 is a flowchart that describes a method of imaging a biological specimen.

FIG. 6 is a block diagram illustrating components of a machine.

### DETAILED DESCRIPTION

Certain biological specimen incubation techniques can be used to help determine a presence or other characteristic of a target substance, such as a target foreign body, within a biological specimen. For example, a plurality of individual biological specimens can be placed into an incubator, each for a respective incubation period. During the incubation period, a target foreign body within an individual biological specimen can become increasingly detectable. Following incubation, the biological specimens can be removed and assayed using one or more specific detection techniques.

One approach to biological specimen incubation involves visual inspection of biological specimens for signs of an infection. This can include observing for signs of a change in color, clarity, size, or other characteristics of the specimen. Here, “visual” inspection can refer to inspection of characteristics visible to a human observer at wavelengths visible to the human eye. Such visual inspection can present certain challenges. It can be difficult to inspect the entire specimen surface, such as to determine a presence of an infectious agent or other foreign body. Also, and manual visual inspection may be subject to possible operator-introduced errors. For example, such an approach can involve manual intervention, such as manual agitation of a sample, during incubation or in between incubation sessions. Such manual intervention can present challenges, such as difficulty in obtaining repeatable results, fluctuating temperatures within the incubator, and possible operator-introduced errors.

In another approach to biological specimen incubation, a sensor can be used to obtain some of the information necessary for determining the presence or other characteristic of the target foreign body within the biological specimen. However, certain approaches involving a sensor can increase the time required to perform the incubation as compared with some manual techniques. Further, using such sensors can require a relatively high level of expertise to configure and operate the system and can be relatively expensive and difficult to scale up.

This document describes, among other things, an automated biological specimen imaging unit, such as for retrofitting into an incubator, that can help address at least some of the challenges of other approaches such as discussed above. Further, an automated biological specimen imaging unit can help provide monitoring of an individual specimen vessel at more frequent intervals or at more accurate timing than is feasible with manual monitoring of multiple specimen vessels.

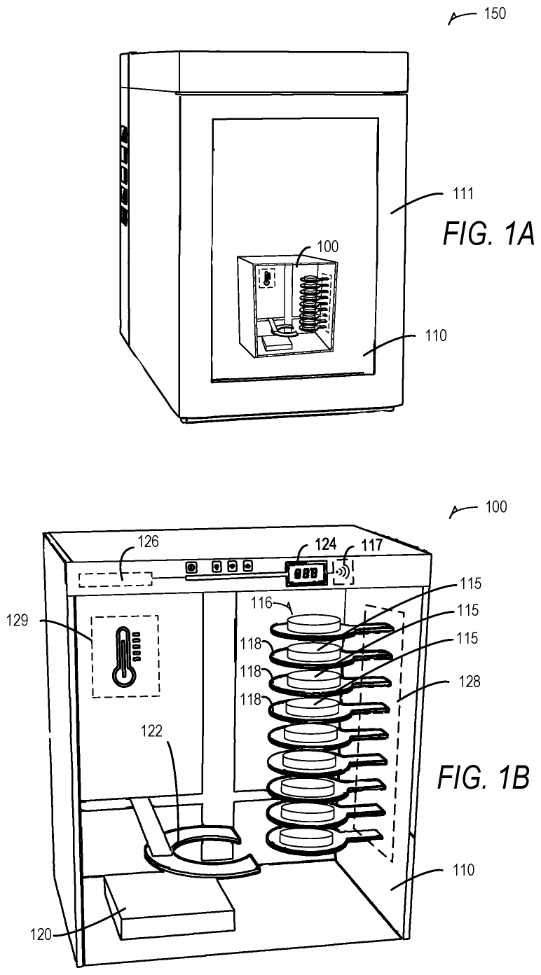

FIG. 1A depicts an example of a biological specimen incubator 150. FIG. 1B depicts a biological specimen imaging unit 100 for placement into a sealed region of the incubator 150 of FIG. 1A. The incubator 150 can include an incubator chamber 110, which can be accessed, e.g., via a door 111. In an example, the imaging unit 100 can include or use at least one assemblage or stack 116, such as including one or more shelves 118. An individual shelf 118 can include one or multiple receptacles for respectively receiving a respective biological specimen vessel. The biological specimen imaging unit 100 can be placed into the chamber 110, such as to introduce biological specimen vessels 115 (depicted in FIG. 1B) into the incubator 150. For example, the specimen imaging unit 100 can be securable within the incubator 150, such as via a coupler or docking mechanism. For example, the coupler can include a fastener, a pin, or other mechanism for attaching the imaging unit 100 to the incubator housing.

The shelves 118 or receptacles can be sized and shaped such as to accept respective biological specimen vessels 115. Examples of a biological specimen vessel 115 can include, e.g., a Petri dish, a multiwell plate, a microtiter plate, a chip, a slide, a tube, such as a tube formed from a heat sealable plastic, a strip, a pad, or other suitable container that holds at least one specimen of a biological material. Once the imaging unit 100, carrying the vessels 115, is introduced into the chamber 110, the incubator chamber 110 can be fluidly sealed off from an ambient environment, thereby enclosing the vessels 115 within the chamber 110.

The imaging unit 100 can also include an imaging or other sensor 120, arranged to be placed inside the incubator in communication with one or more biological specimen vessels for generating a corresponding specimen image of a corresponding biological specimen. In an example, the imaging sensor 120 can include a camera. The camera can be used to generate a digital representation of an individual specimen image. The digital representation can be processed to generate image data for further storage or display. The specimen image can be processed and analyzed to provide quantitative information on the biological material in the vessel(s). For example, the processing and analysis can include one or more of determining cell and/or pathogen counts, cell or nucleus morphometry, cell size measurements, growth rate measurements, or other suitable specimen monitoring techniques. The signal produced by the imaging or other sensor 120 can be signal-processed or analyzed, e.g., based on an electro-chemical detection characteristic or an electro-optical detection characteristic measured by the imaging or other sensor 120 within the chamber 110. For example, the imaging or other sensor 120 can include an imaging array of photosensitive elements configured to detect electromagnetic energy within the specified wavelength band, e.g., visible light or the near-infrared spectrum. Also, for example, the imaging or other sensor 120 can include a solid-state imaging array of detector pixels that can be configured to detect specific scattering, fluorescence emissions, or other optical signatures from the target biological specimen. Other examples of imaging sensors 120 can include electro-chemical sensing systems configured to detect the presence of a gas component or gas components in the specimen fluid. These can be used to sense the presence or other characteristic of a target gas component of interest or a gas component that affects the optical or electro-chemical signatures from the target biological specimen. In an example, the at least one imaging or other sensor 120 can include a plurality of gas sensors each respectively positionable or locatable in communication with a respective vessel 115 or receptacle of the plurality of receptacles. The imaging or other sensor 120 can also include an illuminator, e.g., a broadband or tunable wavelength electromagnetic energy source, arranged to illuminate with electromagnetic energy the individual biological specimen vessel.

At least one of: a) an individual biological specimen vessel 115 or b) the imaging or other sensor 120 can be movable with respect to the other, such as via a manipulator or transporter 122 of the imaging unit 100. The manipulator 122 can place at least one of: a) an individual biological specimen vessel 115 or b) the imaging or other sensor 120 in communication with the other in a field of view. This can help permit imaging of the respective specimen using the imaging or other sensor 120. The manipulator 122 can include a robotic mechanism such as a gantry, an articulating arm, or an articulated platform. The manipulator 122 can include at least one placement sensor for providing feedback in placement using the manipulator 122. For example, the at least one placement sensor can include an optical sensor, and the placement feedback can include one or more LEDs or other light sources emitting energy and one or more photodetectors that detect the emitted energy. The at least one placement sensor can also include one or more capacitive sensors, conductive sensors, IR sensors, or RF sensors, cameras, or combinations thereof.

The at least one imaging or other sensor 120 can generate and transmit a signal that can include information representing a measurement of a concentration or other characteristic of a specified gas component or composition associated with a target biological specimen in an individual one of the specimen vessels 115. A reading of the measurement from the imaging or other sensor 120 can be communicated to and provided at a location outside of the incubator 150, such as at a user interface (UI) 124. For example, the UI 124 can include a display to display the result of the measurement, e.g., the type or concentration of a target gas component detected by the imaging or other sensor 120, or an interpretation of the measurement, e.g., the presence or concentration of a target substance in the vessel 115 represented by the sensor detection. Also, the UI 124 can include other output means, e.g., a speaker, a speaker unit, a vibrator, a buzzer, or other similar output devices. The imaging unit 100 can include transceiver circuitry 117 for transmitting the electrical response signal or the determined presence or other characteristic to a location outside the chamber 110 of the incubator 150. For example, the transceiver circuitry 117 can include an external receiver and a wired or wireless communication link to a device that is external to the chamber 110, such as to a local or remote computer system that can be used to perform computational analysis. Also, for example, the transceiver circuitry 117 can include a communication link to an external device that can be used to perform additional processing to that performed by onboard processing circuitry, such as control of the temperature or pressure of the gas environment contained within the chamber 110.

The imaging unit 100 can include a thermostat 129 to help regulate a temperature of the gas environment contained within the chamber 110. The thermostat 129 can include, e.g., an analog, thermistor, or thermocouple type temperature sensor or electronic temperature sensor configured to determine a temperature. The temperature sensor can be communicatively coupled with a heating and cooling unit of the incubator 150 such as to help control the temperature of the gas environment within the chamber 110. In an example, the heating and cooling unit can include a plurality of heating elements configured to heat a gas environment to a desired temperature. In an example, the heating and cooling unit can include a cooling element, e.g., a Peltier cooling element, to cool the gas environment to a desired temperature. Thus, in an example, the imaging unit 100 can include a thermostat configured to communicate with the incubator 150 such as to maintain a desired temperature by activating one or more heating elements when a temperature of the chamber 110 is below a specified level and activating one or more cooling elements when a temperature of the chamber 110 is above the specified level.

The imaging unit 100 can include an agitator 128, e.g., coupled to the assemblage or stack 116 for moving the stack 116 and agitating the biological specimen vessels carried thereon. For example, the agitator 128 can be used to move liquid carried in the vessel, thereby causing movement in the biological specimen. The agitator 128 can include, e.g., one or more of a vibrating agitator, a rocking agitator, a reciprocal linear agitator, a reciprocating linear agitator, or a reciprocating rotary agitator, any of which can be operated by a motor or by manual operation. For example, the reciprocating linear agitator can include a motor for rotating a cam of the reciprocating linear agitator, such as a motor with an elongated rod and an eccentric weight at its end. In another example, a motor can be used to reciprocate the linear agitator along its length. Alternatively or additionally, the manipulator 122 can be used for agitating, such as to agitate an individual specimen vessel 115.

Processing circuitry 126 can be included or used, such as onboard the imaging unit 100. The processing circuitry 126 can be configured to help control or position the at least one imaging or other sensor 120 relative to the target biological specimen in the specimen vessel 115, or to regulate the position of the at least one manipulator 122. The processing circuitry 126 can include a processor circuit and a memory circuit that can store a program or a series of programs for instructing the processor to carry out the processing steps. For example, the programmed steps can be used such as to establish or adjust a selected temperature and pressure condition within the chamber 110, or to detect the presence or other characteristic of the target gas component in the biological specimen. The processing circuitry 126 can also be used to perform or coordinate other steps, e.g., such as for transmitting the resulting measurement to the UI 124, controlling agitation via the agitator 128, regulating temperature via the thermostat 129, operating the manipulator 122, or communicating via the transceiver circuitry 117. The processing circuitry 126 can include a plurality of processors and memory circuits for individually executing and storing the programs. The processing circuitry 126 can include an external interface that permits communication with devices, computers, and other programs that are external to the biological specimen incubator 150. In an example, the external interface can be couplable to a device external to the incubator 150 via a wired connection, such as a universal standard bus (USB) connection. Here, the wired connection can be fed through an access port of the incubator 150, and the access port can form a seal around the wired connection such as via a gasket or O-ring.

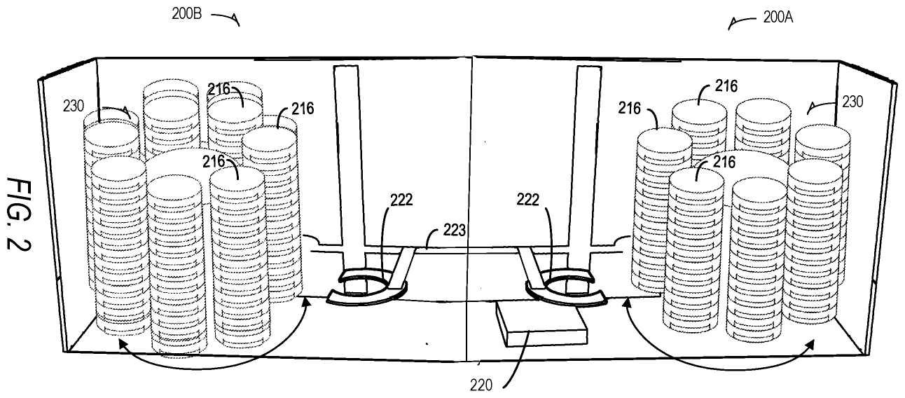

FIG. 2 depicts an example of a modular biological specimen imaging unit for placement within a biological specimen incubator. An individual imaging unit 200, such as imaging unit 200A or imaging unit 200B, can be substantially similar to imaging unit 100 of the example of FIG. 1B. The components, structures, configuration, functions, etc. of imaging unit 200A or imaging unit 200b can therefore be the same as or substantially similar to that described in detail above with reference to imaging unit 100. In an example, an individual biological specimen stack 216 can include a canister or other assemblage including a plurality of biological specimen vessels. Here, biological specimen vessels can be loaded or assembled into a biological specimen stack, e.g., by a technician before the stack is inserted into an imaging unit 200 or an incubator.

In an example, an individual imaging unit 200A can be arranged such that multiple biological specimen stacks 216 can be brought into a field of view of a single imaging or other sensor 220. For example, a carousel 230 can be included such as to cycle a plurality of biological specimen stacks 216 towards or away from a manipulator 222 of the imaging unit 200A. As depicted in FIG. 2, a carousel 230 can be arranged such as to present an individual specimen stack 216 to the manipulator 222, and the manipulator 222 can select or obtain an individual specimen vessel from the stack 216. In an example, the manipulator 222 can select an entire specimen stack 216 from a plurality of specimen stacks 216 without the need of the carousel 230 or similar supplemental mechanism.

In an example, one or more additional imaging units, such as a second imaging unit 200B can be used with or coupled to the imaging unit 200A. As depicted, the two imaging units 200A and 200B can share a common single imaging or other sensor 220. For example, each of the imaging units 200A and 200B can include respective pluralities of stacks 216 and respective manipulators. Also, the two imaging units 200A and 200B can share a common single manipulator 222, such as accessing a common track 223 therebetween.

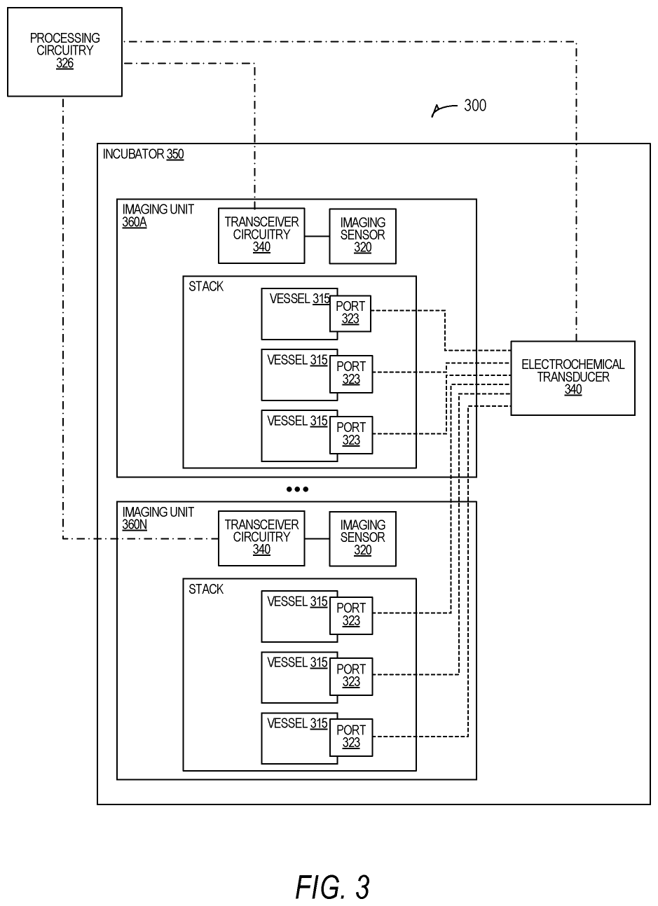

FIG. 3 is a block diagram that describes an example of portions of a biological specimen imaging system. A biological specimen imaging system 300 can include an incubator 350, at least one imaging unit 360A, and a plurality of biological specimen vessels 315. As depicted by the diagram including ellipses in FIG. 3, several imaging units, 360A-360N, can be included in a single incubator 350. Imaging units 360A-360N can be substantially similar to imaging unit 100 of the example of FIG. 1B or imaging unit 200A or 200B of the example of FIG. 2. The components, structures, configuration, functions, etc. of imaging units 360A-360N can therefore be the same as or substantially similar to that described in detail above with reference to imaging unit 100, imaging unit 200A, or imaging unit 200B. An individual biological specimen vessel 315 can also include or be communicatively coupleable to an electrochemical transducer 340 for transducing an electrical property, indicative of a target gas composition corresponding with the particular biological specimen, into an electrical response signal.

For example, a plurality of the biological specimen vessels 315 can include an onboard electrochemical transducer incorporated within the vessel. Thus, an individual specimen vessel 315 can be available for electrochemical transduction, such as gas sensing, concurrent with imaging of the respective vessel 315 via an imaging or other sensor 330. Here, the onboard electrochemical transducer can include transceiver circuitry to transmit the electrical response signal to a location outside the incubator 350. Also, as depicted in FIG. 3, the biological specimen imaging system 300 can include an electrochemical transducer 340 arranged to interface with at least one of the individual specimen vessels 315, such as via a port 323 of an individual vessel 315. For example, the individual specimen vessel 315 can include the respective port 323 sized and shaped such as to be communicatively coupled with a corresponding receptacle of the electrochemical transducer 340. Once connected to the port 323, the electrochemical transducer 340 can transduce an electrical property, indicative of a target gas composition corresponding with the particular biological specimen, into an electrical response signal. An individual specimen vessel 315 can also be connected, such as via the respective port 323, to an optical sensor for obtaining an optical property indicative of a target gas composition corresponding with the particular biological specimen, in a similar fashion to that described above with respect to connection with the electrochemical transducer 340.

As depicted in FIG. 3, the biological specimen imaging system can include processing circuitry 326, such as at a location outside the incubator 350, for processing an image obtained by the imaging or other sensor 320, for processing an electrical response signal obtained by the electrochemical transducer, or both. For example, the processing circuitry 326 can receive the image from transceiver circuitry 317 concurrent with receiving the electrical response signal from the electrochemical transducer 340. Such concurrent or simultaneous imaging and image-transduction of the biological specimen can help provide improved time efficiency relative to, e.g., separately transducing and imaging biological specimens in multiple individual vessels in separate sequential experiments. Also, the processing circuitry 326 can help facilitate conditional imaging, via the imaging or other sensor 320, and based on the electrical response signal. For example, the processing circuitry 326 can facilitate retrieval and processing of a specimen image upon detection of an electrical response signal indicative of a target gas composition. The processing circuitry 326 can also help facilitate conditional electrochemical transduction based on image processing of a specimen image in a similar fashion.

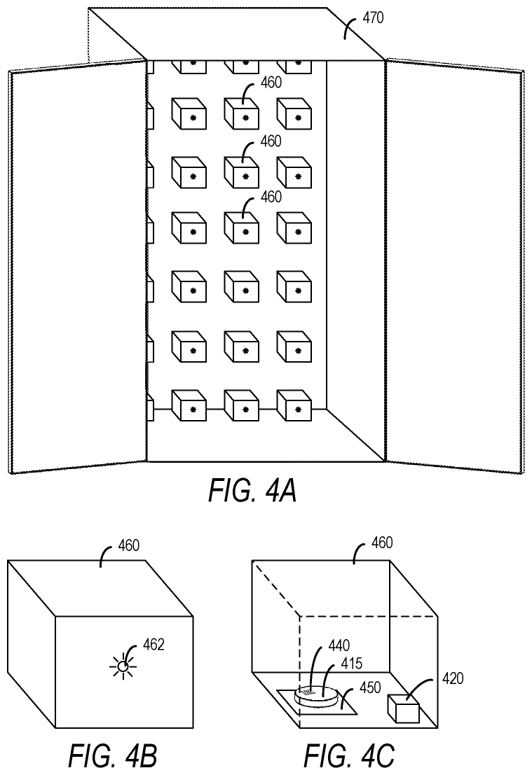

FIG. 4A, FIG. 4B, and FIG. 4C depict an example of a biological specimen imaging system. As shown in FIG. 4A, a plurality of imaging units 460 can be arranged within a cabinet 470. Imaging units 460 can be substantially similar to imaging unit 100 of the example of FIG. 1B, imaging unit 200A or 200B of the example of FIG. 2, or imaging units 360A-360N of the example of FIG. 3. The components, structures, configuration, functions, etc. of imaging units 460 can therefore be the same as or substantially similar to that described in detail above with reference to imaging unit 100, imaging unit 200A, imaging unit 200B, or imaging units 360A-360N. Here, an individual imaging unit 460 can act as a miniature incubator for each individual biological specimen vessel 415 (e.g., a petri dish). As shown in FIG. 4B, an imaging unit 460 can include temperature control, independent of other imaging units 460, via a controllable heating element 450 within a chamber of the imaging unit 460. At least one of the specimen vessel 415 or the imaging unit 460 can include an electrochemical transducer 440, such as for gas sensing. As shown in FIG. 4B, an individual imaging unit 460 can include an indicator 462, such as an LED, for indicating, via visual indicia, the detection or presence of a target gas. An individual imaging unit 460 can also include an imaging or other sensor 420. Alternatively or additionally, the cabinet 470 can include an imaging or other sensor, such as moveable with respect to the imaging units 460 via a manipulator. Here the moveable imaging or other sensor can navigate, e.g., a track system to interface with individual imaging units 460, such as to image biological specimen contained therein. In an example, an individual imaging unit 460 can include a port sized and shaped to mate with a corresponding receptacle of the cabinet 470 such as to enable direct interfacing with a component within included in an individual imaging unit, e.g., an electronic or colorimetric gas sensor, a temperature controller, or a heating element.

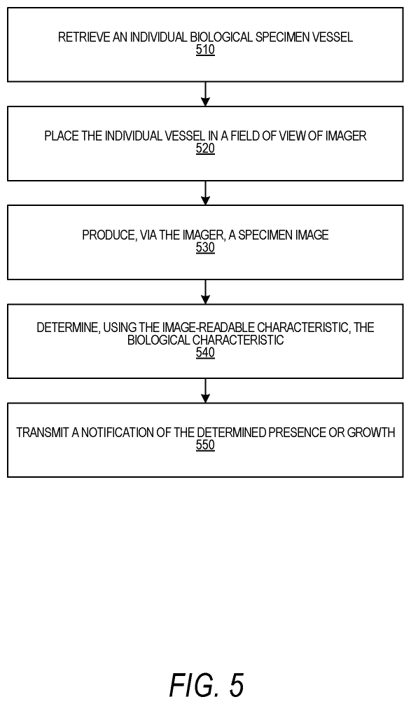

FIG. 5 is a flowchart that describes an example of portions of a method of imaging a biological specimen.

At 510, the method can include retrieving an individual biological specimen vessel from an arrangement defining a plurality of vessel receptacles. The biological specimen vessel can carry a particular biological specimen. The arrangement can be located within an incubated environment and isolated from an ambient environment. Also, the incubating of the individual biological specimen can be concurrent with the producing a specimen image.

At 520, the method can include placing the individual vessel in a field of view of an imager. For example, an individual biological specimen vessel can be retrieved, e.g., by a biological specimen vessel manipulator. The biological specimen vessel manipulator (e.g., a carousel) can then be used to align the individual vessel with the imager. Once the vessel is properly aligned, the imager can be used to capture an image of the contents of the vessel. The carousel can then be used to rotate the vessel, allowing the imager to capture images of the vessel from multiple angles. Finally, the carousel can be used to remove the vessel from the field of view of the imager.

At 530, the method can include producing, via the imager, a specimen image including an image-readable characteristic indicative a biological characteristic associated with the particular biological specimen carried by the vessel. Also, the method can include electrochemically transducing, concurrent with the producing the specimen image, an electrical property indicative of a target gas composition of the particular biological specimen into an electrical response signal.

At 540, the method can include determining, using the image-readable characteristic, the biological characteristic associated with the particular biological specimen. This can include, for example, measuring enzyme activity, analyzing cell morphology, counting cells, assessing cell viability, or other methods. Additionally, this can include techniques such as colorimetry or AI-based image processing to determine the growth of the target substance during incubation. Furthermore, the method can include comparing the biological characteristic to a pre-determined threshold, or to the biological characteristic of a control sample. This can be done to help determine the efficiency of the sample, or to identify any potential abnormalities.

At 550, the method can include transmitting a notification, such as via transceiver circuitry, of the determined presence or growth to a location outside the incubator.

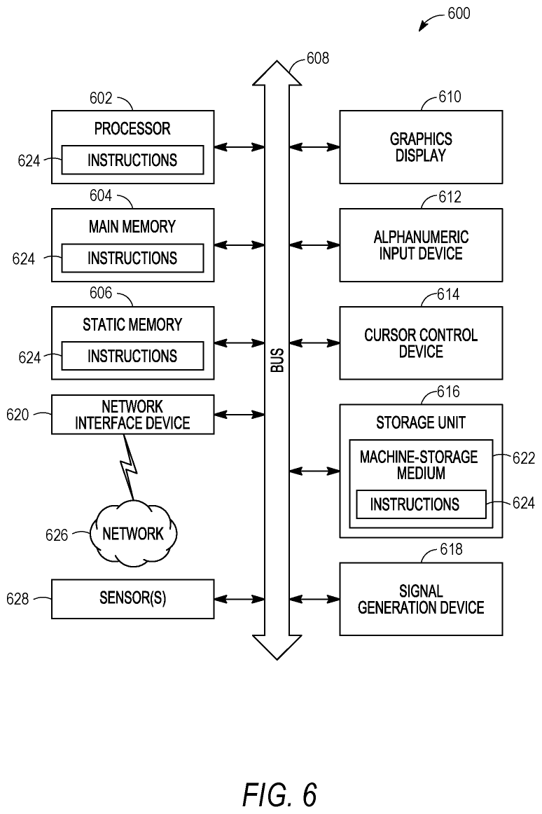

FIG. 6 is a block diagram illustrating components of a machine 600, according to some example embodiments, able to read instructions 624 from a machine-storage medium 622 (e.g., a non-transitory machine-storage medium, a machine-storage medium, a computer-storage medium, or any suitable combination thereof) and perform any one or more of the methodologies discussed herein, in whole or in part. Specifically, FIG. 6 shows the machine 600 in the example form of a computer system (e.g., a computer) within which the instructions 624 (e.g., software, a program, an application, an applet, an app, or other executable code) for causing the machine 600 to perform any one or more of the methodologies discussed herein can be executed, in whole or in part. For example, the instructions 624 can be processor executable instructions that, when executed by a processor of the machine 600, cause the machine 600 to perform the operations outlined above.

In various embodiments, the machine 600 operates as a standalone device or can be communicatively coupled (e.g., networked) to other machines. In a networked deployment, the machine 600 can operate in the capacity of a server machine or a client machine in a server-client network environment, or as a peer machine in a distributed (e.g., peer-to-peer) network environment. The machine 600 can be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any machine capable of executing the instructions 624, sequentially or otherwise, that specify actions to be taken by that machine. Further, while only a single machine is illustrated, the term “machine” shall also be taken to include any collection of machines that individually or jointly execute the instructions 624 to perform all or part of any one or more of the methodologies discussed herein.

The machine 600 includes a processor 602 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 604, and a static memory 606, which are configured to communicate with each other via a bus 608. The processor 602 can contain microcircuits that are configurable, temporarily or permanently, by some or all of the instructions 624 such that the processor 602 is configurable to perform any one or more of the methodologies described herein, in whole or in part. For example, a set of one or more microcircuits of the processor 602 can be configurable to execute one or more modules (e.g., software modules) described herein.

The machine 600 can further include a graphics display 610 (e.g., a plasma display panel (PDP), a light emitting diode (LED) display, a liquid crystal display (LCD), a projector, a cathode ray tube (CRT), or any other display capable of displaying graphics or video). The machine 600 can also include an alphanumeric input device 612 (e.g., a keyboard or keypad), a cursor control device 614 (e.g., a mouse, a touchpad, a trackball, a joystick, a motion sensor, an eye tracking device, or other pointing instrument), a storage unit 616, an audio generation device 618 (e.g., a sound card, an amplifier, a speaker, a headphone jack, any suitable combination thereof, or any other suitable signal generation device), and a network interface device 620.

The storage unit 616 includes the machine-storage medium 622 (e.g., a tangible and non-transitory machine-storage medium) on which are stored the instructions 624, embodying any one or more of the methodologies or functions described herein. The instructions 624 can also reside, completely or at least partially, within the main memory 604, within the processor 602 (e.g., within the processor's cache memory), or both, before or during execution thereof by the machine 600. Accordingly, the main memory 604 and the processor 602 can be considered machine-storage media (e.g., tangible and non-transitory machine-storage media). The instructions 624 can be transmitted or received over the network 626 via the network interface device 620. For example, the network interface device 620 can communicate the instructions 624 using any one or more transfer protocols (e.g., Hypertext Transfer Protocol (HTTP)).

In some example embodiments, the machine 600 can be a portable computing device, such as a smart phone or tablet computer, and have one or more additional input components (e.g., sensors 628 or gauges). Examples of the additional input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components can be accessible and available for use by any of the modules described herein.

### Executable Instructions and Machine-Storage Medium

The various memories (i.e., 604, 606, and/or memory of the processor(s) 602) and/or storage unit 616 can store one or more sets of instructions and data structures (e.g., software) 624 embodying or utilized by any one or more of the methodologies or functions described herein. These instructions, when executed by processor(s) 602 cause various operations to implement the disclosed embodiments.

As used herein, the terms “machine-storage medium,” “device-storage medium,” “computer-storage medium” (referred to collectively as “machine-storage medium 622”) mean the same thing and can be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 622 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage medium or media, computer-storage medium or media, and device-storage medium or media 622 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and can be used interchangeably in this disclosure. The terms are defined to include both machine-storage media and signal media. Thus, the terms include both storage devices/media and carrier waves/modulated data signals.

The following, non-limiting examples, detail certain aspects of the present subject matter to solve the challenges and provide the benefits discussed herein, among others. The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following aspects, the terms “including” and “comprising” are open-ended, that is, a system, device, article, composition, formulation, or process that can include elements in addition to those listed after such a term in a aspect are still deemed to fall within the scope of that aspect.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following aspect, the terms “including” and “comprising” are open-ended, that is, a system, device, article, composition, formulation, or process that can include elements in addition to those listed after such a term in a aspect are still deemed to fall within the scope of that aspect. Moreover, in the following aspects, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the aspects. Also, in the above Detailed Description, various features can be grouped together to streamline the disclosure. This should not be interpreted as intending that an unclaimed disclosed feature is essential to any aspect. Rather, inventive subject matter can lie in less than all features of a particular disclosed embodiment. Thus, the following aspects are hereby incorporated into the Detailed Description as examples or embodiments, with each aspect standing on its own as a separate embodiment, and it is contemplated that such embodiments can be combined with each other in various combinations or permutations. The scope of the invention should be determined with reference to the appended aspects, along with the full scope of equivalents to which such aspects are entitled.

What is claimed is:

1. An apparatus for biological specimen sensing retrofitting inside a separate incubator, the apparatus comprising:
an imager, arranged to be placed inside the incubator with one or more biological specimen vessels for generating a corresponding specimen image of a corresponding biological specimen;
a plurality of receptacles, arranged to be placed inside the incubator, an individual receptacle sized and shaped for receiving an individual one of the vessels;
processing circuitry, configured to control the imager to generate the specimen image, including:
controlling at least one of the imager and the individual vessel to locate the vessel in a field of view of the imager for obtaining the specimen image; and
image-processing the specimen image to determine an indication of a biological characteristic associated with the biological specimen; and
transceiver circuitry for transmitting the indication of the biological characteristic to an interface device located outside the incubator.

2. The apparatus of claim 1, comprising at least one of a vessel transporter or an imager transporter communicatively coupled with the processing circuitry to move at least one of the imager or an individual biological specimen vessel with respect to the other.

3. The apparatus of claim 2, comprising the vessel transporter including a robotic manipulator for retrieving the individual vessel and placing the vessel in a field of view of the imager.

4. The apparatus of claim 2, comprising the imager transporter configured to move the imager towards an individual receptacle of the plurality of receptacles.

5. The apparatus of claim 1, comprising an illuminator, including at least one of a broadband or tunable wavelength electromagnetic energy source, arranged to illuminate with electromagnetic energy the individual biological specimen vessel.

6. The apparatus of claim 1, wherein:
the processing circuitry is configured to generate, for the individual vessel, a sequence of specimen images captured at different times; and
the transceiver circuitry is configured to transmit the sequence of specimen images to a location outside the incubator for monitoring of the individual biological specimen.

7. The apparatus of claim 1, wherein the transceiver circuitry includes wired connection circuitry between the apparatus and a location outside the incubator for providing a wired communication between the imager and a remote receiver.

8. A system for biological specimen sensing inside an incubator, the system comprising:
an incubator including a sealed chamber isolated from an ambient environment; and
at least one imaging unit arranged within the sealed chamber, an individual imaging unit comprising:
an imager, arranged to be placed inside the incubator with one or more biological specimen vessels for generating a corresponding specimen image of a corresponding biological specimen;
a plurality of receptacles, arranged to be placed inside the incubator, an individual receptacle sized and shaped for receiving an individual one of the vessels;
processing circuitry, configured to control the imager to generate the specimen image, including:
controlling at least one of the imager and the individual vessel to locate the vessel in a field of view of the imager for obtaining the specimen image; and
image-processing the specimen image to determine an indication of a biological characteristic associated with the biological specimen; and
transceiver circuitry for transmitting the indication of the biological characteristic to an interface device located outside the incubator.

9. The system of claim 8, comprising the individual vessel, wherein the individual vessel includes an electrochemical transducer for transducing an electrical property, indicative of a target gas composition corresponding with the biological specimen, into an electrical response signal.

10. The system of claim 8, comprising:
an electrochemical transducer for transducing an electrical property, indicative of a target gas composition corresponding with the biological specimen, into an electrical response signal; and
the individual vessel, wherein the individual vessel includes a port configured to be communicatively coupled with a receptacle of the electrochemical transducer.

11. The system of claim 8, comprising:
an optical sensor for an optical property indicative of a target gas composition corresponding with the biological specimen; and
the individual vessel, wherein the individual vessel includes a port sized and shaped to fluidly connect with the optical sensor.

12. The system of claim 8, wherein the at least one imaging unit comprises a plurality of imaging units arranged within the sealed chamber, the plurality of imaging units configured for imaging concurrent with one another.

13. An apparatus for biological specimen sensing inside an incubator, the apparatus comprising:
an imager, arranged to be placed inside the incubator with one or more biological specimen vessels for generating a corresponding specimen image of a corresponding biological specimen;
a first specimen stack defining a first plurality of receptacles arranged to be placed inside the incubator, an individual receptacle sized and shaped for receiving an individual one of the vessels;
a second specimen stack defining a second plurality of receptacles arranged to be placed inside the incubator, an individual receptacle sized and shaped for receiving an individual one of the vessels; and
processing circuitry configured to:
place the imager and a first vessel, received from the first plurality of receptacles, in communication with each other, the first vessel configured for carrying a first particular biological specimen; and
place the imager and a second vessel, received from the second plurality of receptacles, in communication with each other, the second vessel configured for carrying a second particular biological specimen; and
determine, using a specimen image corresponding to at least one of the first vessel or the second vessel, an indication of a biological characteristic associated with the respective first or second particular biological specimen; and
transceiver circuitry for transmitting the indication of the biological characteristic to an interface device located outside the incubator.

14. The apparatus of claim 13, comprising at least one of a vessel transporter or an imager transporter communicatively coupled with the processing circuitry to move at least one of the imager or an individual biological specimen vessel with respect to the other.

15. The apparatus of claim 14, comprising the vessel transporter including a robotic manipulator for retrieving the individual vessel and placing the vessel in a field of view of the imager.

16. The apparatus of claim 14, comprising the imager transporter configured to move the imager towards an individual receptacle of the plurality of receptacles.

17. A method for biological specimen sensing inside an incubator, the method comprising:
retrieving an individual biological specimen vessel from an arrangement defining a plurality of vessel receptacles, the vessel configured for carrying a particular biological specimen;
placing the individual vessel in a field of view of an imager;
producing, via the imager, a specimen image including an image-readable characteristic indicative a biological characteristic associated with the particular biological specimen carried by the vessel;
determining, using the processor to analyze the image-readable characteristic, the biological characteristic associated with the particular biological specimen; and
transmitting a notification of the determined presence or growth to a location outside the incubator.

18. The method of claim 17, comprising isolating the arrangement within an incubated environment and from an ambient environment.

19. The method of claim 17, comprising incubating the individual biological specimen concurrent with the producing a specimen image.

20. The method of claim 17, comprising electrochemically transducing, concurrent with the producing the specimen image, an electrical property indicative of a target gas composition of the particular biological specimen into an electrical response signal.

21. A system comprising:
a chassis;
one or more biological specimen chambers, an individual biological specimen chamber attachable to the chassis and individually temperature controllable, and including at least one of:
an inlet gas port and an outlet gas port;
a sensor for detecting biological specimen growth;
an internal imager or imaging viewport through which an external imager images inside the individual specimen chamber;
an internal illumination device or illumination viewport through which an external illuminator provides illumination; and
processing circuitry for supplying an individual biological specimen chamber with ambient gas.
